(19) United States
(12) Patent Application Publication
(10) Pub. No.: US 2023/0039069 A1
(43) Pub. Date: Feb. 9, 2023

(54) WELL PLATE FOR 3D CELL SPHEROID CULTURE, METHOD FOR MANUFACTURING WELL PLATE FOR 3D CELL SPHEROID CULTURE, AND METHOD FOR 3D CELL SPHEROID CULTURE USING SAME

(71) Applicant: POTECH RESEARCH AND BUSINESS DEVELOPMENT FOUNDATION, Pohang-si (KR)

(72) Inventors: Seong-Jin LEE, Pohang-si (KR); Dong-Sung KIM, Pohang-si (KR); Andrew CHOI, Daegu (KR); Seong-Su EOM, Busan (KR); Jae-Seung YOON, Pohang-si (KR); Sang-Min PARK, Busan (KR)

(21) Appl. No.: 17/790,310

(22) PCT Filed: Feb. 11, 2020
(86) PCT No.: PCT/KR2020/001908

§ 371 Date: Jun. 30, 2022

(30) Foreign Application Priority Data
Jan. 31, 2020 (KR) 10-2020-0011832

(51) Int. Cl. C12M 1/32; C12M 1/12; C12M 1/00; C12M 1/34; C12N 5/00

(52) U.S. Cl. CPC C12M 23/12; C12M 23/04; C12M 23/20; C12M 29/04; C12M 41/12; C12N 5/00; C12N 2513/00

(57) ABSTRACT

The present disclosure relates to a well plate for 3D cell spheroid culture that facilitates 3D cell spheroid culture and enables cell adhesion and detachment by adjusting a surface roughness, a method for manufacturing the well plate for 3D cell spheroid culture, and a method for 3D cell spheroid culture using the same.

### TECHNICAL FIELD

The present disclosure relates to a well plate for a 3D cell spheroid culture, a method for manufacturing the well plate for a 3D cell spheroid culture, and a method for a 3D cell spheroid culture using the same.

### BACKGROUND ART

An ultimate goal of research of a cell culture platform is to realize cells cultured in vitro on the cell culture platform as similarly as possible to cells in tissues in an actual human body. However, cells cultured in vitro in two-dimensions as a monolayer do not reflect the behavior of cells interacting in three-dimensions in an actual human body, and thus are not suitable for use in drug development or regenerative medicine.

In order to overcome limitations of the two-dimensional culture, there is provided a well plate suitable for 3D cell aggregation culture such as 3D spheroids, as in Korean Patent No. 1949856. The well plate suitable for 3D cell aggregation culture induces aggregation of single cells in a partitioned microspace to induce the formation of the 3D cell spheroids.

However, the 3D cell spheroids formed in the well plate as described above should be detached from the well plate and utilized after several days or several weeks culturing in order to be utilized in the field of drug development or regenerative medicine. Therefore, in a well plate according to the related art, a physical external force is applied to 3D cell spheroids by pipetting in a process of detaching the cultured 3D cell spheroids. However, since the pipetting process is performed by manpower, the well plate (microwell) according to the related art is limited in use because it is not suitable in the process of simultaneously detaching a large amount of 3D cell spheroids.

### DISCLOSURE

### Technical Problem

An aspect of the present disclosure is to provide a well plate for 3D spheroid culture with a temperature-sensitive surface roughness change that implements induction of formation of single cells into 3D cell spheroids, which is an advantage of a well plate for 3D cell spheroid culture according to the related art, and maintains characteristics suitable for culture at a cell culture temperature, and enables a large amount of 3D cell spheroids to be simultaneously detached from the well plate due to a change in surface roughness properties at room temperature, a method for manufacturing the well plate, and a method for 3D cell spheroid culture and detachment using the same.

### Technical Solution

According to an aspect of the present disclosure, a well plate for 3D cell spheroid culture includes a well plate chamber including a porous microwell at a lower portion, wherein a cell culture layer in which cells adhere at higher than 32° C. to 40° C. or lower and the cells are detached at 0° C. or higher to lower than 32° C. is formed on an upper surface of the porous microwell.

According to another aspect of the present disclosure, a method for manufacturing a well plate for 3D cell spheroid culture includes: providing an aqueous coating solution containing an N-isopropylacrylamide monomer, a cross-linking agent, and a balance of water, in which the cross-linking agent is contained in an amount of 1 part by weight or more to less than 5 parts by weight with respect to 100 parts by weight of a mixture of the N-isopropylacrylamide monomer and the water; forming a coating layer using the aqueous coating solution on an upper surface of a porous microwell of a well plate chamber including the porous microwell; and forming a cell culture layer by irradiating the coating layer with UV rays.

According to still another aspect of the present disclosure, a method for 3D cell spheroid culture includes adding a cell culture medium and cells to a well plate chamber in the well plate for cell spheroid culture, and culturing the cells.

### Advantageous Effects

As set forth above, the well plate having a surface structure with a surface roughness suitable for cell culture and harvest through a change in temperature is provided, such that a well plate that is advantageous for mass production and harvest of 3D cell spheroids may be provided.

### DESCRIPTION OF DRAWINGS

FIG. 1 is a view illustrating an example of a well plate for cell spheroid culture manufactured by an exemplary embodiment in the present disclosure.

FIG. 2 illustrates an image of an upper chamber including a porous microwell manufactured by an exemplary embodiment in the present disclosure when observed from above. FIG. 2B is a view obtained by enlarging one porous microwell among a plurality of porous microwells of FIG. 2A.

FIG. 3 illustrates a change in surface roughness of a cell culture layer according to a temperature and a time in an exemplary embodiment in the present disclosure.

FIG. 4A is a view obtained by enlarging the porous microwell, and FIG. 4B illustrates that a cell culture layer containing polyisopropylacrylamide is formed on an upper surface of the porous microwell.

FIG. 5A illustrates an image captured in a process of culturing 3D cell spheroids in the well plate for cell spheroid culture manufactured according to an exemplary embodiment in the present disclosure. FIG. 5B illustrates cell spheroids detached from the well plate for spheroid culture. FIG. 5C is a view obtained by capturing an image of the well plate for spheroid culture after the cell spheroids are detached.

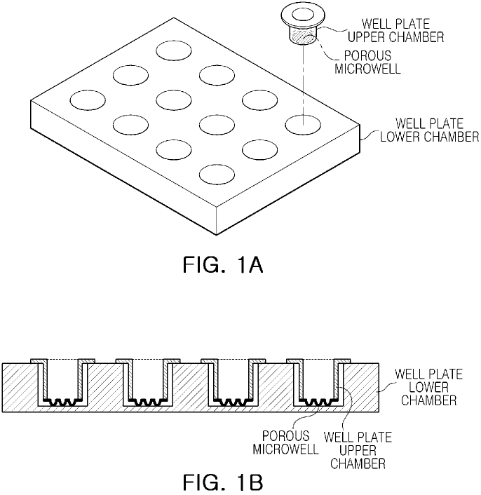

### BEST MODE FOR INVENTION

Hereinafter, exemplary embodiments in the present disclosure will be described with reference to the accompanying drawings. However, exemplary embodiments in the present disclosure may be modified in several other forms, and the scope of the present disclosure is not limited to exemplary embodiments to be described below.

A cell culture layer included in a well plate for 3D cell spheroid culture according to the related art is not suitable for mass-producing and harvesting 3D spheroids because it does not have a function of adhesion and detachment of the 3D cell spheroids.

In order to solve this problem, the present disclosure provides a well plate for 3D cell spheroid culture with a temperature-sensitive surface structure change, the well plate including a cell culture layer capable of implementing adhesion and detachment of 3D cell spheroids according to a change in temperature.

Specifically, the present disclosure may provide a well plate for 3D cell spheroid culture including a well plate chamber including a porous microwell at a lower portion, wherein a cell culture layer in which cells adhere at higher than 32° C. to 40° C. or lower and the cells are detached at 0° C. or higher to lower than 32° C. is formed on an upper surface of the porous microwell.

The porous microwell may be formed by random entanglement of a plurality of polymer nanofibers or may be formed by molding a polymer synthetic resin. Since the porous microwell is formed of a plurality of polymer nanofibers, the porous microwell may have a plurality of pores formed in a region between the polymer nanofibers. In this case, the pore may have a size of tens of nm to several µm. That is, the porous microwell may serve as a selective permeable membrane through which other substances selectively permeate without permeation of single cells due to the pores. Therefore, the porous microwell may serve as a substance transfer barrier and passage. For example, the porous microwell may have a pore size with an average diameter of 100 nm to 20 µm, and preferably may have a pore size with an average diameter of 100 nm to 5 µm.

In this case, the polymer nanofiber or the polymer synthetic resin may include at least one of a thermoplastic resin, a thermosetting resin, an elastomer, and a biopolymer. For example, the polymer nanofiber or the polymer synthetic resin may include at least one of polycaprolactone, polyurethane, polyvinylidene fluoride (PVDF), polystyrene, collagen, gelatin, and chitosan, but the present disclosure is not limited thereto.

The porous microwell includes a cell culture layer that is concave in a downward direction, which is a form that is suitable for 3D cell spheroid culture, and the cell culture layer is entirely or partially formed of the polymer nanofiber or the polymer synthetic resin, such that the porous microwell may have the pores described above.

The upper surface (cell culture layer) of the porous microwell is a region in which cells are cultured, and may have a downwardly concave section. The cells may be easily settled by the concave section of the microwell, and may be stably cultured in the porous microwell. Therefore, the single cells may be assembled in a specific region of the porous microwell in the concave section of the microwell, and the assembled single cells may be aggregated into 3D cell spheroids.

Furthermore, the present disclosure may provide a well plate for 3D cell spheroid culture including a cell culture layer whose surface structure is changed according to a temperature by coating a coating composition with a surface structure change according to a temperature onto the porous microwell.

The cell culture layer with the surface structure change according to a temperature may be formed on an upper surface of the porous microwell, and provide a surface structure suitable for cell culture due to its high Adhesive force at higher than 32° C. to 40° C. or lower, and provide a surface structure suitable for collecting the cells due to its surface structure suitable for detachment of cells at 0° C. or higher to lower than 32° C.

The cell culture layer may contain polyisopropylacrylamide (poly(N-isopropylacrylamide) (PNIPAAm)) and may have a surface roughness change characteristic according to a temperature, in which a surface roughness may be 4 nm to 37 nm and preferably 20 nm to 32 nm at higher than 32° C. to 40° C. or lower, and a surface roughness may be 4 µm or more at 0° C. or higher to lower than 32° C. In this case, the surface roughness is measured with a non-contact atomic force microscope (Park Systems, Korea) using a PPP-NCHR cantilever using a frequency of 300 kHz or a BL-AC40TS cantilever using a frequency of 25 kHz.

The cell culture layer may contain a cross-linking agent in an amount of 1 to 5 wt% and preferably more than 1 wt% to less than 3 wt% with respect to the total weight of the cell culture layer. When the content of the cross-linking agent is less than 1 wt%, the cells may not adhere to the cell culture layer well, and when the content of the cross-linking agent is more than 5 wt%, the cell spheroids may not be detached well at a temperature lower than a lower critical solution temperature (LCST).

In addition, the porous microwell has permeability to a fluid, while the rest area of the well plate chamber except the porous microwell area does not have permeability to a fluid. In this case, a porosity of the porous microwell may have a pore size with an average diameter of 100 nm to 20 µm, and preferably may have a pore size of 100 nm to 5 µm. The porous microwell may serve as a selective permeable membrane through which other substances selectively permeate without permeation of single cells due to such a pore size. Therefore, the porous microwell may serve as a substance transfer barrier and passage.

In addition, in a case in which a fluid flows through the porous microwell and the cell culture layer due to a difference in permeability between the porous microwell and its periphery as described above, a fluid concentration phenomenon may occur in the porous microwell. When such a fluid concentration phenomenon occurs, oxygen and nutrients contained in the fluid may be smoothly supplied to cells proliferating and differentiating in the porous microwell. Accordingly, cell proliferation and differentiation may be further promoted. In addition, a phenomenon in which the cells are assembled into the porous microwell also occurs due to such a fluid concentration phenomenon, which facilitates formation of cell spheroids. Therefore, 3D cell spheroids may be easily produced.

In addition, the well plate of the present disclosure may include the well plate chamber as a well plate upper chamber, and a well plate lower chamber in which the well plate upper chamber may be disposed. The lower chamber may be a chamber in which an upper chamber is disposed or mounted to allow a cell culture medium and the like to flow. Furthermore, in the present disclosure, in order to simultaneously culture several cells, the well plate for cell spheroid culture may use a lower chamber in which a plurality of well plate upper chambers may be disposed, and the number of the upper chambers may not be limited as long as it is the number commonly used in the art.

Furthermore, the present disclosure provides a method for manufacturing a well plate for 3D cell spheroid culture.

Specifically, there is provided a method for manufacturing a well plate for 3D cell spheroid culture, the method including: providing an aqueous coating solution containing an N-isopropylacrylamide monomer, a cross-linking agent, and a balance of water, in which the cross-linking agent is contained in an amount of 1 part by weight or more to less than 5 parts by weight with respect to 100 parts by weight of a mixture of the N-isopropylacrylamide monomer and the water; forming a coating layer using the aqueous coating solution on an upper surface of a porous microwell of a well plate chamber including the porous microwell at a lower portion; and forming a cell culture layer by irradiating the coating layer with UV rays.

In the present disclosure, an adhesion and detachment rate of cells according to a change in temperature of a support for cell culture may be controlled by adjusting the content of the cross-linking agent. As described above, the cross-linking agent may be contained in an amount of 1 part by weight or more to less than 5 parts by weight and preferably more than 1 part by weight to less than 3 parts by weight with respect to 100 parts by weight of the mixture of the N-isopropylacrylamide monomer and the water. When the content of the cross-linking agent is less than 1 part by weight with respect to 100 parts by weight of the mixture of the N-isopropylacrylamide monomer and the water, the cells may not adhere well to the support for cell culture, and when the content of the cross-linking agent is 5 parts by weight or more, the cells may not be detached well at a temperature lower than the LCST.

Meanwhile, the cross-linking agent serves to polymerize an N-isopropylacrylamide monomer into polyisopropylacrylamide, and any cross-linking agent may be used without limitation as long as it may be used in a common method used for preparation of polyisopropylacrylamide, that is, homopolymerization, copolymerization, terpolymerization, cross-linked polymerization, or the like. N,N'-Methylenebisacrylamide (MBAAm), tetramethylethylenediamine (TEMED), or a mixture thereof may be preferably used as the cross-linking agent.

The aqueous coating solution may further contain a photoinitiator so that the monomers are cross-linked by UV rays in the aqueous coating solution. In this case, the photoinitiator may be contained in an amount of 0.01 to 0.1 parts by weight and preferably 0.01 to 0.05 parts by weight with respect to 100 parts by weight of the mixture of the N-isopropylacrylamide monomer and the water. When the content of the photoinitiator is less than 0.01 parts by weight, cross-linking by UV rays may not be performed, and when the content of the photoinitiator is more than 0.05 parts by weight, cells may die in a subsequent cell culture due to toxicity of the cross-linking agent itself.

Meanwhile, any photoinitiator may be used without limitation as long as it may initiate cross-linking by UV rays, and for example, 2-hydroxy-1-1-[4-(hydroxyethoxy)phenyl]-2-methyl-1-propanon e may be used as the photoinitiator.

The forming of the coating layer is not limited as long as a coating layer obtained using the aqueous coating solution is formed to the extent that the fluid permeates into the porous microwell. For example, in the forming of the coating layer, the coating layer may be formed using spin coating or bar coating.

Further, since the cell culture layer formed by the coating layer is formed in a form such as hydrogel, the solution moves smoothly. Therefore, a fluid may flow through the porous microwell and the hydrogel.

The forming of the cell culture layer by irradiating the coating layer with UV rays is a step of polymerizing a monomer, and is not limited as long as irradiation with UV rays is performed to the extent that a polymer of N-isopropylacrylamide (polyisopropylacrylamide) is formed. For example, the forming of the cell culture layer may be performed by irradiation with UV rays at 1,800 w for 10 minutes.

The well plate for 3D cell spheroid culture formed as described above may facilitate adhesion and detachment of cells according to a temperature. More detailed description and operation of the cell culture layer have already been described in the well plate for 3D cell culture described above, and thus will be omitted below.

In addition, the method for manufacturing a well plate for 3D cell spheroid culture of the present disclosure may further include disposing a well plate upper chamber in a well plate lower chamber using the well plate chamber as the well plate upper chamber. The detailed description and operation of the lower chamber have already been described in the well plate for 3D cell culture described above, and thus will be omitted below.

Further, the present disclosure may provide a method for 3D cell spheroid culture, the method including adding a cell culture medium and cells to the well plate for 3D cell spheroid culture manufactured by the manufacturing method of the present disclosure or the well plate upper chamber of the well plate for 3D cell spheroid culture of the present disclosure, and culturing the cells.

When 3D cell spheroids are cultured, the well plate upper chamber may be filled with a cell culture medium for cell culture, the cell culture medium may flow through the porous microwell of the well plate upper chamber to the well plate lower chamber together with the flow of the fluid described above to cause a fluid concentration phenomenon. The cell culture medium may contain materials required for cell culture, and is not limited as long as it is a culture medium used in the art.

However, the cell culture medium needs to be replaced periodically for a reason such as a change in pH due to cell growth, waste accumulation, or exhaustion of nutrients. In this case, the cell culture medium may be replaced by a method of supplying a new fluid into the well plate upper chamber while performing suction and discharge of the fluid to the outside of the well plate from well plate lower chamber through the permeability of the porous microwell and the cell culture layer to the fluid. This is because, in a case in which the fluid is directly sucked and discharged from the inside of the well plate upper chamber, there is a risk of adversely affecting the cells settling in the porous microwell and are proliferating and differentiating or a risk that the cells are sucked and discharged together with the fluid.

Accordingly, the culturing of the cells is performed by including performing suction and discharge of the cell culture medium in the well plate lower chamber to the outside of the well plate and adding the cell culture medium to the well plate upper chamber, such that a new cell culture medium may be added to the cells during the cell culturing.

Meanwhile, for adhesion and detachment of cells to be cultured, the method for 3D cell spheroid culture may further include culturing 3D cell spheroids by allowing the 3D cell spheroids to adhere to the cell culture layer at a temperature of higher than 32° C. to 40° C. or lower, and detaching the 3D cell spheroids from the cell culture layer at a temperature of 0° C. or higher to lower than 32° C., and the cells may be myoblasts, embryo fibroblasts, human umbilical vein endothelial cells, or human epidermal cells, but are not limited thereto.

Hereinafter, the present disclosure will be described in more detail with reference to specific Examples. The following Examples are only examples provided in order to assist in the understanding of the present disclosure, but the scope of the present disclosure is not limited thereto.

### MODE FOR INVENTION

### Example 1

A well plate upper chamber including a porous microwell at a lower portion was manufactured. Specifically, holes with a certain size were drilled in polymethyl methacrylate (PMMA), and polymer nanofibers were combined with the perforated PMMA together with an adhesive, thereby manufacturing a porous microwell.

Then, an aqueous solution of N-isopropylacrylamide having a high content ratio and an aqueous solution of N-isopropylacrylamide having a low content ratio were prepared using a phase separation phenomenon occurring in a solution containing a high concentration of an N-isopropylacrylamide monomer. A well plate for 3D cell spheroid culture in which a cell culture layer in which cells adhered to an upper surface of a porous microwell at higher than 32° C. to 40° C. or lower and the cells were detached at 0° C. or higher to lower than 32° C. was formed on the upper surface of the porous microwell of the manufactured well plate upper chamber including the porous microwell using the aqueous coating solution was manufactured.

Specifically, an N-isopropylacrylamide monomer and distilled water were mixed at a mass ratio of 1:1, and stirring was performed for 5 minutes so that the N-isopropylacrylamide monomer was sufficiently dissolved in the distilled water. The N-isopropylacrylamide aqueous solution at a low concentration and the N-isopropylacrylamide aqueous solution at a high concentration were stably divided over time, and when the N-isopropylacrylamide aqueous solutions were finally stably separated, each of the solutions was transferred to a vial using a pipette, thereby obtaining 5 ml of an aqueous solution of N-isopropylacrylamide having a high content ratio in which a mass ratio of N-isopropylacrylamide:water was 87:13.

Then, 0.05 g of N,N'-methylenebisacrylamide (MBAAm) (1 part by weight with respect to 100 parts by weight of the mixture of the N-isopropylacrylamide monomer and the water) as a cross-linking agent for a reaction to UV rays during a UV irradiation treatment and 0.005 g of 2-hydroxy-1-1-[4-(hydroxyethoxy)phenyl]-2-methyl-1-propanon e as a photoinitiator (0.01 parts by weight with respect to 100 parts by weight of the mixture of the N-isopropylacrylamide monomer and the water) were added to each of the N-isopropylacrylamide aqueous solutions.

The composition prepared by adding the cross-linking agent and the photoinitiator to the N-isopropylacrylamide aqueous solution was thinly applied onto the polymer nanofibers using bar coating, and the composition was irradiated with a UV light source for 10 minutes, thereby manufacturing a well plate for 3D cell spheroid culture in which a cell culture layer in which cells adhered at higher than 32° C. to 40° C. or lower and the cells were detached at 0° C. or higher to lower than 32° C. was formed on an upper surface of a porous microwell.

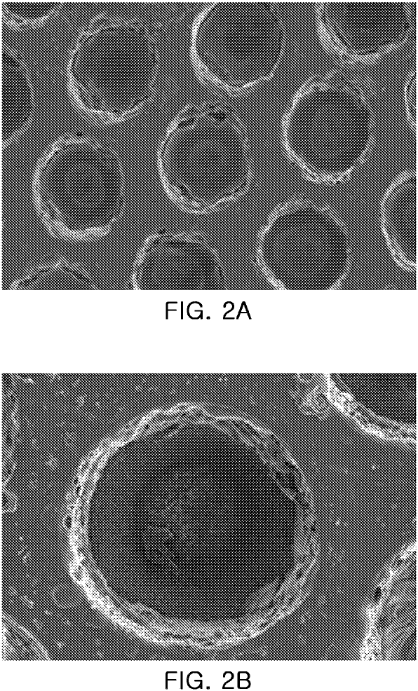

FIGS. 2A and 2B illustrate the well plate for 3D cell spheroid culture in which a cell culture layer in which cells adhered at higher than 32° C. to 40° C. or lower and the cells were detached at 0° C. or higher to lower than 32° C. was formed on the upper surface of the manufactured porous microwell.

### Experimental Example 1

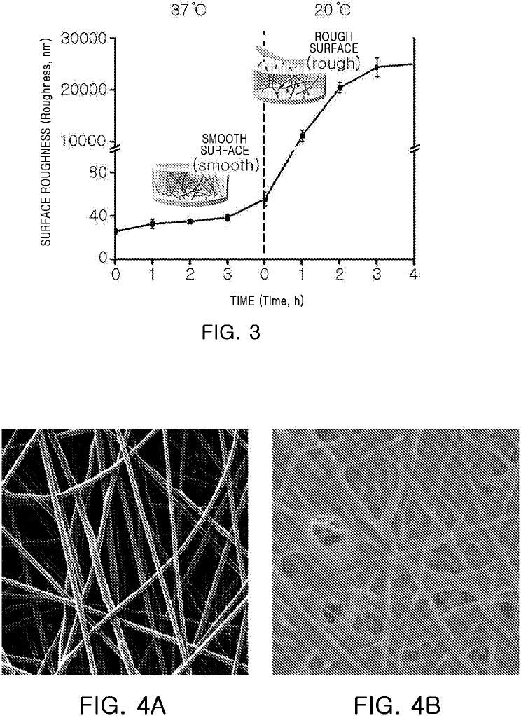

In order to measure a change in surface roughness, a change in surface roughness according to the temperature of the cell culture layer of Example 1 and the time was measured with an atomic force microscope (Park systems, Korea). The measured results are illustrated in FIG. 3.

As illustrated in FIG. 3, it could be confirmed that the surface roughness of the cell culture layer of Example 1 was hardly changed at 37° C., which was a temperature higher than the LSCT, whereas the surface roughness of the cell culture layer of Example 1 was rapidly changed at 20° C., which was a temperature of lower than the LSCT.

### Experimental Example 2

In the plate for 3D cell culture manufactured in Example 1, in order to check a morphological difference of the cell culture layer in which the cells adhered at higher than 32° C. to 40° C. or lower and the cells were detached at 0° C. or higher to lower than 32° C., the surface shapes were compared using a scanning electron microscope.

FIG. 4A illustrates the composition containing N-isopropylacrylamide before coated to the polymer nanofibers, and FIG. 4B illustrates the cell culture layer after the composition containing N-isopropylacrylamide is coated to the polymer nanofibers. The cell culture layer has a form in which the polymer nanofibers and hydrogel are combined.

### Experimental Example 3

A human liver cancer cell line (HepG2) was seeded in the well plate for cell culture of Example 1 at 36° C., and 3D cell spheroid culture was performed after 3 days. In order to harvest the cultured 3D human liver cancer cell line spheroids, the upper chamber containing the 3D human liver cancer cell spheroids was transferred to an environment of 20° C.

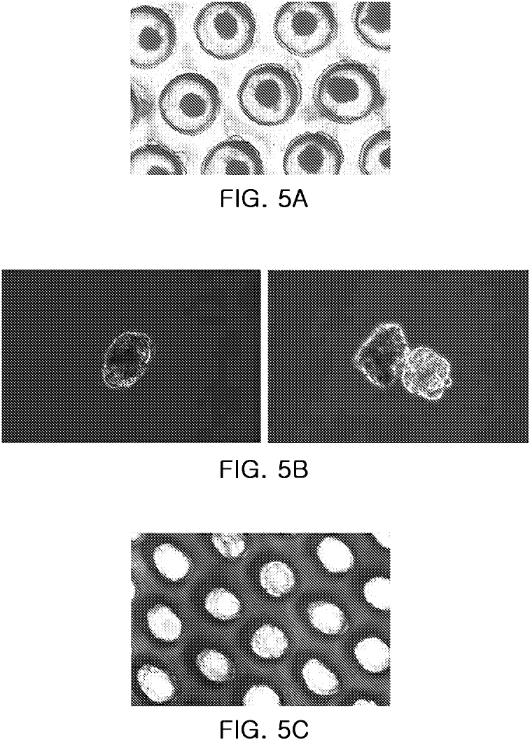

FIG. 5A is a captured image of the well plate in which cells are being cultured, and FIG. 5B illustrates a captured image of the cultured cells. In addition, FIG. 5C illustrates a captured image of the well plate from which the cultured cells are detached.

As a result, as illustrated in FIG. 5C, it could be confirmed that the cells cultured in the well plate for cell culture of the present disclosure were easily detached.

Although exemplary embodiments in the present disclosure have been described in detail above, it will be apparent to those skilled in the art that the scope of the present disclosure is not limited thereto, but various modifications and variations could be made without departing from the technical idea of the present disclosure described in the claims.

1. A well plate for 3D cell spheroid culture comprising a well plate chamber including a porous microwell at a lower portion,
wherein a cell culture layer in which cells adhere at higher than 32° C. to 40° C. or lower and the cells are detached at 0° C. or higher to lower than 32° C. is formed on an upper surface of the porous microwell.

2. The well plate for 3D cell spheroid culture of claim 1, wherein the cell culture layer contains polyisopropylacrylamide (poly(N-isopropylacrylamide) (PNIPAAm)), and has a surface roughness of 4 to 37 nm at higher than 32° C. to 40° C. or lower and a surface roughness of 4 µm or more at 0° C. or higher to lower than 32° C.

3. The well plate for 3D cell spheroid culture of claim 2, wherein the cell culture layer contains a cross-linking agent in an amount of 1 to 5 wt% with respect to a total weight of the cell culture layer.

4. The well plate for 3D cell spheroid culture of claim 3, wherein the cross-linking agent is N,N'-methylenebisacrylamide (MBAAm), tetramethylethylenediamine (TEMED), or a mixture thereof.

5. The well plate for 3D cell spheroid culture of claim 1, wherein pores of the porous microwell have pore sizes of 100 nm to 20 µm in average diameter.

6. The well plate for 3D cell spheroid culture of claim 1, wherein the well plate for 3D cell spheroid culture includes the well plate chamber as a well plate upper chamber and a well plate lower chamber in which the well plate upper chamber is disposed.

7. A method for manufacturing a well plate for 3D cell spheroid culture, the method comprising:
providing an aqueous coating solution containing an N-isopropylacrylamide monomer, a cross-linking agent, and a balance of water, in which the cross-linking agent is contained in an amount of 1 part by weight or more to less than 5 parts by weight with respect to 100 parts by weight of a mixture of the N-isopropylacrylamide monomer and the water;
forming a coating layer using the aqueous coating solution on an upper surface of a porous microwell of a well plate chamber including the porous microwell; and
forming a cell culture layer by irradiating the coating layer with UV rays.

8. The method for manufacturing a well plate for 3D cell spheroid culture of claim 7, wherein the aqueous coating solution further contains a photoinitiator, and the photoinitiator is 2-hydroxy-1-[4-(hydroxyethoxy)phenyl]-2-methylpropan-1-one.

9. The method for manufacturing a well plate for 3D cell spheroid culture of claim 8, further comprising disposing a well plate upper chamber in a well plate lower chamber using the well plate chamber as the well plate upper chamber.

10. A method for 3D cell spheroid culture, the method comprising adding a cell culture medium and cells to a well plate upper chamber in the well plate for cell spheroid culture of claim 1 and culturing the cells.

11. The method for 3D cell spheroid culture of claim 10, wherein the culturing of the cells is performed at a temperature of higher than 32° C. to 40° C. or lower.

12. The method for 3D cell spheroid culture of claim 10, further comprising detaching cell spheroids cultured at a temperature of 0° C. or higher to lower than 32° C. after the cell culture is complete.
